(19) United States
(12) Patent Application Publication
(10) Pub. No.: US 2014/0128695 A1
(43) Pub. Date: May 8, 2014

(54) PORTABLE 2-DIMENSION OXIMETER IMAGE DEVICE

(71) Applicant: National Chiao Tung University, Hsinchu (TW)

(72) Inventors: Wai-Chi Fang, Hsinchu (TW); Tien-Ho Chen, Hsinchu (TW); Shih Kang, Hsinchu (TW); Shih-Yang Wu, Hsinchu (TW); Ching-Ju Cheng, Hsinchu (TW)

(73) Assignee: NATIONAL CHIAO TUNG UNIVERSITY, Hsinchu (TW)

(21) Appl. No.: 13/897,845

(22) Filed: May 20, 2013

(30) Foreign Application Priority Data
Nov. 8, 2012 (TW) 101141514

(51) Int. Cl. A61B 5/1455; A61B 5/00

(52) U.S. Cl. CPC A61B 5/14552; A61B 5/7271
USPC 600/323

(57) ABSTRACT

A portable 2-dimension oximeter image device is disclosed. The portable 2-dimension oximeter image device includes a plurality of light sources for emitting light to an object to be measured such that a plurality of sensors can sense the intensity of reflected light from the measured object; an analysis processor for receiving the light intensity sensed by the front-end detectors to analyze and calculate based on an oxygen saturation distribution algorithm to generate oxygen saturation distribution information; an image reconstruction unit for reconstructing an image according to the oxygen saturation distribution information to generate image information with a color scale to demonstrate the differences in the object's oxygen saturation in each regional tissue of the object, thereby providing an effective and accurate regional detection range.

### BACKGROUND OF THE INVENTION

1. Field of the Invention

The present invention relates to an oximeter image technique, and more particularly, to a portable 2-dimension oximeter image device.

2. Description of Related Art

The medical research has pointed out that oxygen saturation can be used as an index of health and survival. Insufficient oxygen carried by hemoglobin in the blood results in that the oxygen saturation is low and the body is harmful. Therefore, in the medical institutions or on personal care, the demand on oximeters for measuring the oxygen saturation value is increased.

Most commercially available oximeters are pulse oximeters, which determine the oxygen saturation based on the absorptive capacity of the hemoglobin at a specific spectrum. The pulse oximeter usually measures a thinner portion of the body, such as a fingertip or earlobe, and light sources having different wavelengths irradiate on the measured portion. When the light passes through the measured portion, part of the light will be absorbed by the erythrocyte, the rest of the light can be sensed by the light sensors, the conventional oximeter may sense light via transmission or reflection to show the hemoglobin concentration and oxygen saturation. FIGS. 1A and 1B are schematic diagrams illustrating the conventional oximeter sensing oxygen saturation using transmission and reflection, respectively. In FIG. 1A, the user's finger is inserted into a measure space of the oximeter. The light source is the light emitting diode 10, and the sensor 11 is disposed at a position opposing to that of the light emitting diode 10, such that light passing through the finger is sensed by the sensor 11. In FIG. 1B, the light source of the oximeter 1 is also the light emitting diode 10. The sensor 11 and the light-emitting diode 10 are disposed at the same side. The reflected light is sensed in FIG. 1B. In the above conventional oximeters (pinch type), only the oxygen saturation information of a single point can be obtained. It is insufficient to judge the oxygen saturation merely based on such information.

Therefore, there is a need to provide a better mechanism for measuring oxygen saturation so as to avoid the defects of the conventional single point measurement, and to provide convenience and instant images, in order to provide a portable oximeter with low cost and instant images.

### SUMMARY OF THE INVENTION

The present invention provides a portable 2-dimension oximeter image device, which has a regional detection range by means of the design of the flexible sensors, a plurality of light sources and sensors.

The present invention provides a real-time oxygen saturation distribution based on the detection result of oxygen saturation via the image reconstruction technique.

The present invention provides a portable 2-dimension oximeter image device, including a front-end detector for detecting oxygen saturation status; an analysis processor for analyzing the sensed data and an image reconstructing unit for reconstructing an image at the back end. The front-end detector has a plurality of light sources and a plurality of sensors. The light sources emit light to an object to be measured and the sensors sense intensity of the reflected light from the measured object. The analysis processor receives the intensity of the reflected light sensed by the front-end detector and analyzes the intensity of the reflected light to generate oxygen saturation distribution information based on an oxygen saturation distribution algorithm. The image reconstructing unit reconstructs an image according to the oxygen saturation distribution information to generate image information with a color scale to demonstrate the differences in the object's oxygen saturation in each regional tissue of the object.

In an embodiment, in the portable 2-dimension oximeter image device, the detection panel is made of flexible printed circuit board.

In another embodiment, the image reconstructing unit of the portable 2-dimension oximeter image device reconstructs the image using interpolation.

In comparison with the conventional technique, the present invention provides a portable 2-dimension oximeter image device, wherein the front-end detector is designed with a flexible printed circuit board, such that the conventional single point detection is improved to be a regional detection, and the detection accuracy is also improved. In addition, the present invention uses image construction technique for reconstructing images based on the sensed oxygen saturation distribution information, such that the displayed image is smoother and the final image is demonstrated with a color scale. The portable 2-dimension oximeter image device not only provides a real-time image, but also allows users to distinguish the oxygen distribution status more easily.

### BRIEF DESCRIPTION OF DRAWINGS

FIGS. 1A and 1B are, respectively, schematic diagrams illustrating the conventional oximeter sensing oxygen saturation using transmission or reflection;

FIG. 2 is a schematic diagram illustrating the portable 2-dimension oximeter image device according to the present invention;

FIG. 3 is a schematic diagram illustrating the portable 2-dimension oximeter image device according to an embodiment of the present invention; and

FIG. 4 is a schematic diagram illustrating light sources and sensors of a front-end detector of the portable 2-dimension oximeter image device according to the present invention.

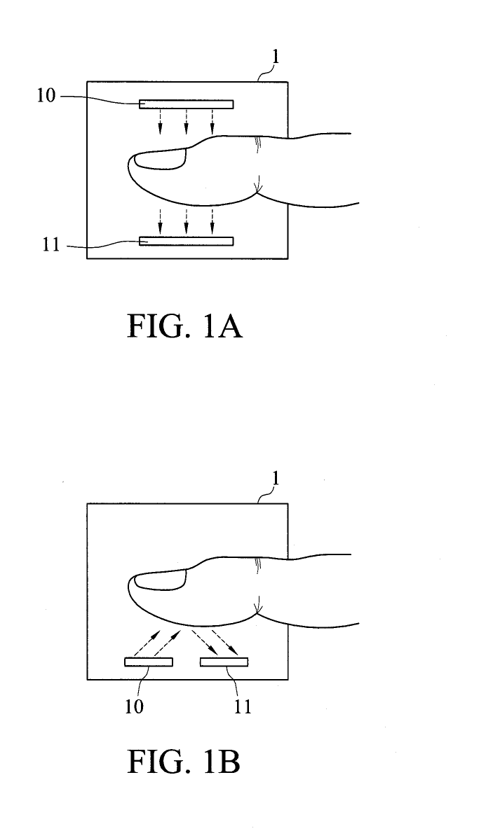

### DETAILED DESCRIPTION OF PREFERRED EMBODIMENTS

The following illustrative embodiments are provided to illustrate the disclosure of the present invention and its advantages, these and other advantages and effects being apparent to those in the art after reading this specification.

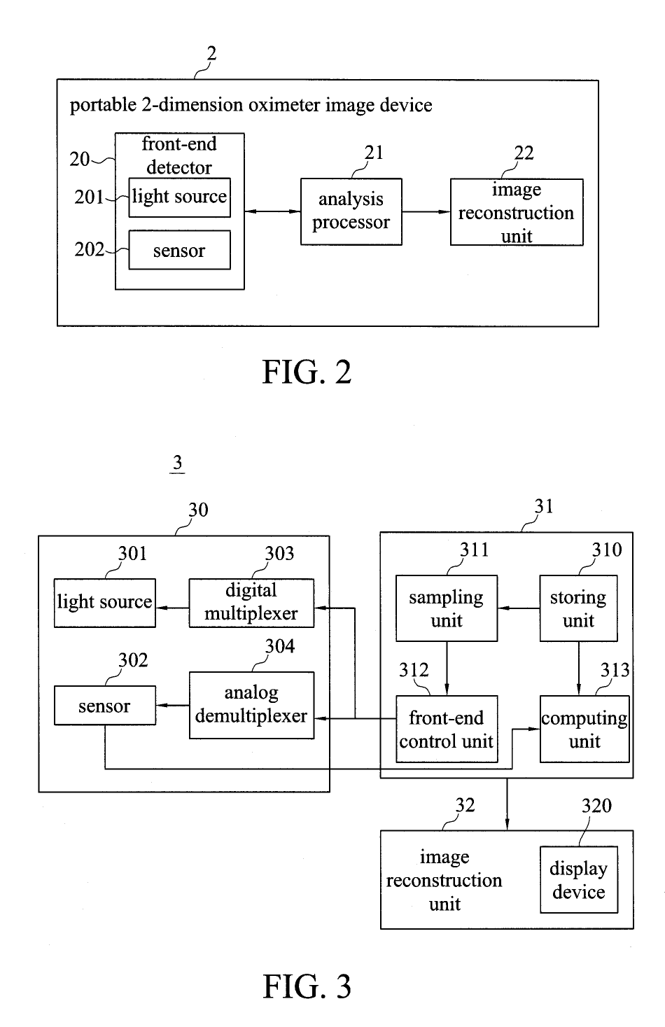

FIG. 2 is a schematic diagram illustrating the portable 2-dimension oximeter image device according to the present invention. FIG. 2 illustrates the internal structure of the portable 2-dimension oximeter image device 2. The portable 2-dimension oximeter image device 2 is used to sense the oxygen saturation of a region of an object to be measured and provide instant image of the oxygen distribution. The object may be many parts of the human body. The portable 2-dimension oximeter image device 2 includes a front-end detector 20, an analysis processor 21 and an image reconstruction unit 22.

The front-end detector 20 has a plurality of light sources 201 and a plurality of sensors 202. The plurality of light sources 201 emit light to an object (not shown), so as to sense the intensity of the reflected light from the object by the plurality of sensors 202. Specifically, the front-end detector 20 is used to sense the oxygen saturation status of the object. When the light emitted by the light sources 201 is sent to the object, the light is reflected after being absorbed by the hemoglobin in the object. The sensors 202 receive the reflected light. The intensity of the reflected light is sensed.

The front-end detector 20 has the plurality of light sources 201 and the plurality of sensors 202, such that sensing data of a plurality of points may be provided. Such data constitute a regional sensing range. The conventional oximeter only uses a set of light source 201 and sensor 202, so as to obtain sensing data of a single point. The portable 2-dimension oximeter image device 2 of the present embodiment can obtain a plurality of accurate oxygen saturation data, and the data also provide the oxygen distribution for imaging.

Further, the light source is a dual wavelength light source, such as red light or infrared light. Since the oxygenated hemoglobin and the non-oxygenated hemoglobin in the object have different absorption coefficients to different wavelengths. Thus, detections of the oxygenated hemoglobin and the non-oxygenated hemoglobin in response to different wavelengths are performed to determine oxygen saturation data. In this embodiment, a dual-wavelength light source is provided at 735 nm and 890 nm. The received wavelength range of the detector is set to 320 nm to 1050 nm. The size of the aforementioned dual wavelength light source may be adjusted based on the needs of operation.

The analysis processor 21 receives the intensity of the reflected light sensed by the front-end detector 20 and analyzes the intensity of the reflected light based on an oxygen saturation distribution algorithm to generate distribution information of the oxygen saturation of each regional tissue of the object. The analysis processor 21 is used for analyzing the information sensed by the front-end detector 20. The information such as the intensity of the reflected light sensed by the sensors 202 is sent to the analysis processor 21. Based on the analysis of the oxygen saturation distribution algorithm, the distribution information of the oxygen saturation of each regional tissue of the object is generated. The aforesaid oxygen saturation distribution algorithm is an algorithm that records the correlation of the intensity of light sources and the oxygen saturation. The data of the oxygen saturation distribution is obtained by analyzing the intensity of the light sources. Since the front-end detector 20 has a plurality of light sources 201 and sensors 202, there are multiple sensing data. The sensing data of a plurality of positions are provided based on the distribution of the light sources 201. Therefore, the oxygen saturation distribution of each of the regional tissues of the object is provided. Specifically, the distribution information of the oxygen saturation may contain the coordinates of each regional tissue of the object and the oxygen saturation values corresponding to the coordinates. That is, the coordinates demonstrate the relationship in position between the plurality of positions and provide the oxygen saturation value of the coordinate. The coordinates and values are used in an image reconstruction unit 22 for subsequent image reconstruction and imaging.

The image reconstruction unit 22 reconstructs the image based on the distribution information of the oxygen saturation generated by the analysis processor 21, in order to generate image information of the oxygen saturation of each regional tissue of the object demonstrated with a color scale. Since the conventional oximeters only provides a single point detection, the sensing results can not be presented as a regional image. However, the portable 2-dimension oximeter image device 2 of the present embodiment has an image reconstruction unit 22 for presenting the regional data into an image. The image reconstruction unit 22 performs image reconstruction for expanding the original screen to facilitate the viewing. Therefore, the image reconstruction unit 22 performs image reconstruction based on the obtained distribution information of the oxygen saturation to generate image information. The image information demonstrates the oxygen saturation of each regional tissue of the object with a color scale, so as to facilitate users to directly view the oxygen saturation distribution images.

The image reconstruction method used in the present embodiment is interpolation, i.e. using the two adjacent pixels and calculating the pixels of the third point between the above two adjacent pixels to gradually expand data amount. For example, with a plurality of oxygen saturation distribution information provided by six light sources and 12 sensors, the original available screen being only 24 pixels is expanded to 273 pixels, which facilitates the user to watch the screen, and the reconstructed image is smoother.

In addition, in order to enhance the practicality of the portable 2-dimension oximeter image device 2, the detection panel of the front-end detector 20 can be made of a flexible printed circuit board. The conventional oximeter is limited to be used in a finger or earlobe. The front-end detector 20 of the portable 2-dimension image device 2 may be made of a flexible or soft material, such as a flexible printed circuit board. Moreover, the front-end detector 20 includes the plurality of light sources 201 and sensors 202 for increasing the detection range and reducing the limitation of positions.

Also, the soft or flexible material facilitates the attachment to various parts of the object, thereby increasing the convenience of the portable 2-dimension oximeter image device 2.

Referring to FIG. 3, FIG. 3 is a schematic diagram illustrating the portable 2-dimension oximeter image device according to an embodiment of the present invention. As shown in FIG. 3, the front-end detector 30 includes a digital multiplexer and an analog demultiplexer 304. The analysis processor 31 includes a storing unit 310, a sampling unit 311, a front-end control unit 312 and a computing unit 313.

The front-end detector 30 is used for detecting the oxygen saturation of the object by using light sources 301 and sensors 302. The front-end detector 30 has the digital multiplexer 303 and the analog demultiplexer 304 for controlling the plurality of light sources 301 and the plurality of sensors 302, respectively. In other words, the digital multiplexer 303 is used for selecting the light sources 301 to be turned on and the analog demultiplexer 304 is used for selecting the corresponding sensors 302 when operating the portable 2-dimension oximeter image device 3. In order to avoid mutual interference, the light sources 301 do not emit the light at the same time, and the plurality of sensors 302 are not simultaneously turned on for detection. Therefore, based on demand of the detection, the portable 2-dimension oximeter image device 3 controls the activation of the light sources 301 and the sensors 302 through the digital multiplexer 303 and the analog demultiplexer 304.

The analysis processor 31 is a processing core of the portable 2-dimension oximeter image device 3, especially for detecting the oxygen saturation and analyzing the sensed value. The internal main components of the analysis processor 31 are described as follows.

The storing unit 310 stores setting parameters concerning detecting and sampling. In order to provide various detections, the detection range of the portable 2-dimension oximeter image device 3 is adjustable. The storing unit 310 stores setting parameters needed for detecting and sampling, wherein the setting parameters may include the intensity of the light sources, the absorption coefficients of oxygenated hemoglobin and the absorption coefficients of oxygenated hemoglobin received by the front-end detector 30. In other words, the setting parameters include the intensity of the light sources which can be received by the sensors 302 of the front-end detector 30. At the same time, it is considerable to adjust the setting parameters containing the absorption coefficients of oxygenated hemoglobin and the absorption coefficients of oxygenated hemoglobin according to the objects to be measured. In other words, the absorption coefficients of oxygenated hemoglobin and the absorption coefficients of oxygenated hemoglobin are changed according to different objects, and thus the sensed intensities of the light sources are different. These parameters affect the oxygen saturation distribution algorithm for calculating the oxygen saturation distribution.

Thus, with the change of the setting parameters, there are different combinations of the front-end detector 30 and the oxygen saturation distribution algorithm in the computing unit 313. Therefore, in the different front-end detectors 30 and the different processors (i.e., different setting parameters), the required oxygen saturation image can be accurately completed.

In addition, the setting parameters also include the sampling rate of the front-end detector 30 and an updating rate of the image information. In other words, the sampling rate of the front-end detector 30 of the portable 2-dimension oximeter image device 3 can be set. The different sampling rates affect the updating status of the following image content, such that the user can set the desired sampling rate. The aforementioned setting parameters can be input, updated and inquired through the user interface.

The sampling unit 311 is used to generate the sampling instructions of the front-end detector 30 based on the setting parameters. The sampling unit 311 acquires the setting parameters in the storing unit 310, to generate the sampling commands for demanding the detection of the front-end detector 30. The sampling commands demand the front-end control unit 312 to transmit a control signal to the front-end detector 30.

The front-end control unit 312 is connected to the front-end detector 30 for transmitting the control signal to the digital multiplexer 303 and the analog demultiplexer 304 of the front-end detector 30 to control operations of the digital multiplexer 303 and the analog demultiplexer 304, such that the operation of the light sources 301 and the sensors 302 are controlled.

The computing unit 313 is used for implementing the oxygen saturation distribution algorithm to calculate the oxygen saturation corresponding to the intensity of the light sources sensed by the plurality of sensors 302. That is, the computing unit 313 computes the oxygen saturation of the sensing region using the oxygen saturation distribution algorithm according to the correlation between the intensity of the light sources and the oxygen saturation. A plurality of data may generate the oxygen saturation distribution information for the image reconstruction unit 32 to perform reconstruction and imaging.

The image reconstruction unit 32 includes the display device 320 for displaying the reconstructed image information. As previously described, in this embodiment, the color scale is used to demonstrate the different oxygen saturation distribution and thus to distinguish the different statuses of the oxygen saturation. For example, the higher oxygen saturation can be demonstrated in red. In addition, in addition to the reconstructed images, the image information may also include the reconstructed images under the intensity of the light sources of wavelength of 735 nm or wavelength of 890 nm In other words, in addition to the reconstructed images generated under the dual wavelength sensing, the respective images of different wavelengths may also be displayed.

In order to provide better practicability of the portable 2-dimension oximeter image device, the related contents may be designed and made with a chip system. The technical contents displayed by the aforementioned two-dimensional oxygen image may be achieved by a VLSI so as to generate a miniaturized device for instantly imaging. The analysis processor 31 may include a decoding processing unit (not shown) and an encoding unit (not shown). The decoding processing unit can be designed to receive the input instructions or setting parameters from the user interface. The decoding processing unit may decode the received setting parameters and store them in the storing unit 310. Alternatively, the received input instruction may be decoded for issuing the corresponding control command to the sampling unit 311, the front-end control unit 312 or the computing unit 313. The encoding unit can be used for encoding the intensity of the light sources. Data of the intensity of the light sources can be provided through the user interface. That is, if the user would like to view data of the sensed intensity of the light sources through the user interface, rather than the reconstructed images, the encoding unit may encode the intensity of the light sources for generating the corresponding data displayed on the user interface.

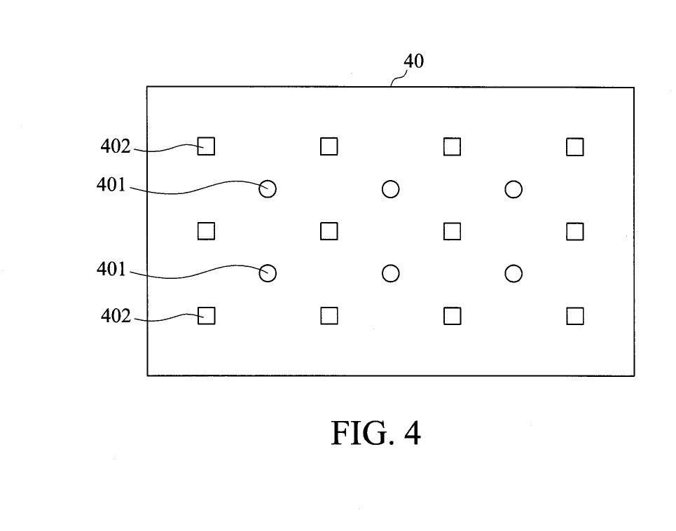

Referring to FIG. 4, FIG. 4 is a schematic diagram of light sources and sensors of a front detector of the portable 2-dimension oximeter image device according to the present invention. As shown in FIG. 4, the front-end detector 40 has a plurality of light sources 401 and sensors 402. In this embodiment, the front-end detector is consisting of six light sources 401 (demonstrated in circular) and twelve sensors 402 (demonstrated in square). That is, there are four sensors 402 around each of the light sources 401. When the distance between any two adjacent sensors 402 is 2 cm, the distance between any two adjacent light sources 401 is also 2 cm, the distance between the light source 401 and the sensor 402 is 1.414 cm, and the reconstructed area can reach 12 cm², i.e., the size of the sensed region is 12 cm². With the digital multiplexer 303 and the analog demultiplexer 304 mentioned in FIG. 3, it can be understood that the light sources 401 and the sensors 402 are not enabled simultaneously, but a certain light source 401 and its surrounding four sensors 402 are enabled based on the sensing range. For example, when an order of detection is given, it is determined which light source 401 is enabled at the first time, and which sensors 402 are enabled to sense. Subsequently, the next light source 401 is enabled, and its surrounding sensors 402 are enabled to sense, and so on. In addition, if there is no mutual interference, several sets of sensors can be enabled simultaneously. The enablement of the light sources 401 and the detection order of the sensors 402 are controlled by the sampling unit 311 and the front-end control unit 312 shown in FIG. 3.

In addition, on the part of the aforesaid image reconstruction, when the front-end detector 40 has six light sources 401 and twelve sensors 402, due to the different positions of the sensors 402 in the front-end detector 40, different amount of data can be sensed. For example, the sensors 402 at the four corners only have one sensing data (since only one light source 401 is needed to be sensed at the surrounding), while the middle sensors 402 have four sensing data (since four light sources 401 are sensed at the surrounding). Therefore, after the sensing is performed once, the obtained data are equivalent to the data obtained by 24 conventional oximeters. Therefore, initial information of 24 pixels is generated. After image reconstruction, it can be extended to be an easy viewing image that is reconstructed by using interpolation.

Compared to the conventional techniques, the present invention provides a portable 2-dimension oximeter image device, wherein the front-end detector has a plurality of light sources and sensors, so as to perform a regional detection, rather than the conventional single point detection, such that the display of regional image rather than single point data is provided by the portable 2-dimension oximeter image device. The displayed image can be expanded and smoothed through the image reconstruction techniques. The different distribution statuses of oxygen saturation are expressed with a color scale, such that the user may understand the oxygen saturation easily. Furthermore, the front-end detector can be made of a soft printed circuit board or flexible material. With the plurality of light sources and sensors, the detection positions may fit the human body, and practicability is enhanced.

The above-described descriptions of the detailed embodiments are to illustrate the preferred implementation according to the present invention, and are not intended to limit the scope of the present invention. Accordingly, many modifications and variations completed by those with ordinary skill in the art will fall within the scope of present invention as defined by the appended claims.

What is claimed is:

1. A portable 2-dimension oximeter image device, including:
a front-end detector having a plurality of light sources and a plurality of sensors, the plurality of light sources emitting light to an object to be measured and the plurality of sensors sensing intensity of reflected light from the object;
an analysis processor receiving the intensity of the reflected light sensed by the front-end detector and analyzing the intensity of the reflected light to generate oxygen saturation distribution information based on an oxygen saturation distribution algorithm; and
an image reconstruction unit reconstructing an image according to the oxygen saturation distribution information to generate image information with a color scale to demonstrate oxygen saturation in each regional tissue of the object.

2. The portable 2-dimension oximeter image device of claim 1, wherein the front-end detector comprises a detection panel made of a flexible printed circuit board.

3. The portable 2-dimension oximeter image device of claim 1, wherein the front-end detector includes a digital multiplexer and an analog demultiplexer for controlling the plurality of light sources and the plurality of sensors respectively.

4. The portable 2-dimension oximeter image device of claim 1, wherein the light sources is a dual wavelength light source.

5. The portable 2-dimension oximeter image device of claim 1, wherein the oxygen saturation distribution information contains coordinate values of each regional tissue of the object and oxygen saturation values corresponding to the coordinate values.

6. The portable 2-dimension oximeter image device of claim 1, wherein the analysis processor comprises:
a storing unit for storing setting parameters concerning detecting and sampling;
a sampling unit for generating sampling instructions of the front-end detector in accordance with the setting parameters;
a front-end control unit connected to the front-end detector for issuing a control signal based on the sampling instructions; and
a computing unit for performing the oxygen saturation distribution algorithm to calculate the oxygen saturation corresponding to the intensity of the reflected light.

7. The portable 2-dimension oximeter image device of claim 6, wherein the analysis processor comprises:
a decoding processing unit for receiving the setting parameters from a user interface to decode the setting parameters and storing decoded results in the storing unit; and
an encoding unit for encoding the intensity of the reflected light to provide data of the intensity of the reflected light by the user interface.

8. The portable 2-dimension oximeter image device of claim 6, wherein, the setting parameters include the intensity of the reflected light, absorption coefficients of oxygenated hemoglobin and absorption coefficients of deoxygenated hemoglobin.

9. The portable 2-dimension oximeter image device of claim 6, wherein the setting parameters include a sampling rate of the front-end detector and an updating rate of the image information.

10. The portable 2-dimension oximeter image device of claim 1, wherein the image reconstructing unit uses interpolation for reconstructing the image information.
